(19) United States
(12) Patent Application Publication
(10) Pub. No.: US 2023/0277702 A1
(43) Pub. Date: Sep. 7, 2023

(54) SYSTEM FOR DISINFECTION APPLICATIONS

(71) Applicant: SIGNIFY HOLDING B.V., EINDHOVEN (NL)

(72) Inventors: MATTHIAS WENDT, WÜRSELEN (DE); PETER DEIXLER, ARLINGTON, MA (US)

(21) Appl. No.: 18/008,208

(22) PCT Filed: May 28, 2021
(86) PCT No.: PCT/EP2021/064309

§ 371 Date: Dec. 5, 2022

Related U.S. Application Data
(60) Provisional application No. 63/034,831, filed on Jun. 4, 2020.

(30) Foreign Application Priority Data
Jun. 11, 2020 (EP) 20179528.3

(51) Int. Cl. A61L 2/10; A61L 2/08; A61L 2/28

(52) U.S. Cl. CPC A61L 2/10; A61L 2/084; A61L 2/28; A61L 2202/11; A61L 2202/14

(57) ABSTRACT

The invention relates to a system, a light unit, a sensor device and a method for disinfection applications. The system (100) comprises a) a light unit (110) comprising i) a light source (111), e.g. LED, adapted to emit at least disinfectant light (115), and ii) a controller (112) for controlling the light source such that at least a part of the light is encoded with information on light characteristics of the disinfectant light, and b) a sensor device (120) comprising i) a detection surface (121) adapted to be arranged on an object (130) and to detect the encoded light, and ii) a disinfection status deriving unit (122) adapted to derive a disinfection status of the object based on the light characteristics encoded in the encoded light. This allows for providing a good estimate of a disinfection status of an object treated with the disinfection light.

### FIELD OF THE INVENTION

The invention relates to a system, a light unit, a sensor device and a method for disinfection applications.

### BACKGROUND OF THE INVENTION

As increased antibiotics resistance of germs is predicted over the next 20 years, infection prevention will become very important in the future. The benefits of episodic cleaning alone are short-lived, since it has been shown that germs almost immediately repopulate a previously cleaned space. Thus, it would be beneficial to provide a system that allows for a continuous preventive disinfection of a predefined area, for instance, a room in a hospital.

Recently, the possibility of using light for disinfection purposes has been further investigated. In this context, it has been shown that light in the ultraviolet spectrum can be used for disinfection purposes, for instance, for disinfecting air, water or medical instruments. However, constant exposure to UV light, in particular, UV-C light, provides health risks for living beings and thus has to be avoided.

Recent developments have shown that, as an alternative to UV light, also light in longer wavelengths, in particular, light in the visible spectrum, preferably around 405 nm, can be used for disinfection purposes, since this light is absorbed by porphyrin molecules inside of bacteria causing the formation of biocidal reactive oxygen. This process allows to suffocate and kill the bacteria and can lead to a reduced contamination when applied continuously to an area. Since light in the visible spectrum does not provide any health risks to living beings, its application for disinfection purposes can be widely increased, for instance, to general hospital rooms, elderly care environments, but also common spaces like schools, sports complexes or airports. Moreover, such light disinfection can also be advantageous for food processing facilities or agriculture and horticulture applications.

A general advantage of light-based disinfection is that the disinfection is not surface specific but works on air, hard and soft surfaces and generally on all places where the light can reach the germs. Thus, using light for disinfection purposes provides many advantages in a plurality of applications. However, a problem with light disinfection, in particular, when applied to common environments like room settings, is that a user cannot know if an object provided in the room is already disinfected or, for instance, if the light that has reached the object has not provided enough intensity or has not been provided over enough time to allow for a significant reduction of the bacteria on the surface of the object. Thus, when applying light for disinfection purposes, the disinfection status of objects is often completely unknown. It would therefore be advantageous to provide a system that allows the application of disinfection light in common room settings by providing a good estimate of the disinfection status of objects within the room setting.

Coded light refers to techniques whereby data is modulated into the light emitted by a light source, such as an LED-based light source. In this way the data may be said to be embedded into the light from the light source. For instance, data may be modulated into the illumination emitted by a luminaire such as an LED-based luminaire. Thus in addition to providing illumination, the light source also acts as a transmitter capable of transmitting data to a suitable receiver of coded light. The modulation is typically performed at a high enough frequency that, despite the illumination being in the visible spectrum, the modulation is imperceptible to human vision. I.e. so the user only perceives the overall illumination and not the effect of the data being modulated into that illumination. Data is modulated into the light by means of a technique such as amplitude keying or frequency shift keying, whereby the modulated property (e.g. amplitude or frequency) is used to represent channel symbols. The modulation typically involves a coding scheme to map data bits (sometimes referred to as user bits) onto such channel symbols. An example is a conventional Manchester code, which is a binary code whereby a user bit of value 0 is mapped onto a channel symbol in the form of a low-high pulse and a user bit of value 1 is mapped onto a channel symbol in the form of a high-low pulse. Another example is the recently developed Ternary Manchester code.

There are a number of known techniques for detecting and decoding coded light at the receive side. For example, coded light can be detected using an everyday ‘rolling shutter’ type camera, as is often integrated into a mobile device like a mobile phone or tablet. In a rolling-shutter camera, the camera's image capture element is divided into a plurality of lines (typically horizontal lines, i.e. rows) which are exposed in sequence line-by-line. That is, to capture a given frame, first one line is exposed to the light in the target environment, then the next line in the sequence is exposed at a slightly later time, and so forth. Typically the sequence ‘rolls’ in order across the frame, e.g. in rows top to bottom, hence the name ‘rolling shutter’. When used to capture coded light, this means different lines within a frame capture the light at different times and therefore, if the line rate is high enough relative to the modulation frequency, at different phases of the modulation waveform. Thus the modulation in the light can be detected. Coded light can also be detected by using a global shutter camera if the frame rate is high enough relative to the modulation frequency, or using a dedicated photocell with suitable sample rate.

### SUMMARY OF THE INVENTION

It is an object of the present invention to provide a system, a light unit, a sensor device and a method which allows to providing a good estimate of a disinfection status of an object treated with the disinfection light.

In a first aspect of the present invention, a system for disinfection applications is presented, wherein the system comprises a) a light unit comprising i) a light source adapted to emit light, wherein at least a part of the emitted light is disinfectant light usable for disinfection purposes, and ii) a controller configured to control the light source, wherein the controller is configured to control the light source such that at least a part of the emitted light is encoded with information on light characteristics of the emitted disinfectant light, and b) a sensor device comprising i) a detection surface adapted to be arranged on an object and to detect emitted light from the light unit, wherein the detection surface is adapted to detect at least the emitted encoded light, and ii) a disinfection status deriving unit adapted to derive a disinfection status of the object based on the light characteristics encoded in the emitted encoded light.

Since the light unit comprises a controller that is configured to control the light source such that at least a part of the emitted light of the light source is encoded with information on light characteristics of the emitted disinfectant light, information necessary for estimating a disinfection status of an object that should be disinfected by the disinfectant light are directly provided together with the disinfectant light. Moreover, since the sensor device is provided that can be arranged on an object and that is adapted to detect at least the emitted encoded light from the light unit, the sensor device is directly provided with knowledge on the disinfectant light falling on the object. Thus, the sensor device can provide a very good estimate of the disinfection status of the object on which it is arranged. The system therefore allows to utilize light for disinfection purposes while at the same time providing a good estimate of the disinfection status of an object that should be disinfected by the disinfectant light.

The light source is adapted to emit light. In particular, the light source is adapted to emit light in a predetermined range of the electromagnetic spectrum, wherein at least a part of the light spectrum emitted by the light source is usable for disinfection purposes. Preferably, a light source is adapted to emit at least disinfectant light in the deep blue and/or purple part of the visible spectrum, more preferably, disinfectant light comprising a wavelength of 405 nm. Additionally or alternatively, the light source can also be adapted to emit disinfectant light in an ultraviolet part of the spectrum, preferably in the UV-C part of the spectrum. Moreover, although not as effective for containing viruses, also light in the infrared spectrum can be employed for disinfection purposes, with respect to certain pathogens. Thus, the light source can also be adapted to emit light in the infrared spectrum as disinfectant light.

Generally, it is known that bacteria and viruses vary somewhat in UV susceptibility with environmental organisms, fungal spores, and mycobacteria being relatively harder to kill than more rapidly replicating and non-environmental microbes and most bacteria. Shorter-wavelength UV photons have higher energy potential than longer-wavelength UV photons, and may have an accelerated aging effect on materials and paints. UV-C photons may even damage plants. Hence, the preferred spectrum to be provided by the light source and used for disinfection purposes can depend on the material present in the room, the material of the object to be disinfected, the presents of living beings in a room, etc., as well as on the required efficacy of killing viruses and germs. Preferably, the light is provided such that the object is exposed to radiation varying from 200 to 1,000 J/m2, i.e. 20 to 100 mJ/cm2 depending on the type of surface and its cleanliness.

The light source can be adapted to emit only the disinfectant light and thus to emit only light in a part of the electromagnetic spectrum that is usable for disinfection purposes. However, the light source can also be adapted to emit light in addition to the disinfectant light, preferably, the additional light is emitted in a visible part of the electromagnetic spectrum and/or in the infrared part of the electromagnetic spectrum.

For emitting the light, the light source comprises at least one light providing device being preferably a light emitting diode (LED). However, it can also comprise other light providing devices like a VCSEL, LASER, or a gas discharge lamp. In an embodiment, the light source comprises only one light providing devices, e.g. LED, that is adapted to emit at least the disinfectant light. However, in another embodiment, the light source comprises at least two light providing devices, e.g. LEDs, wherein one of the light providing devices is adapted to emit the disinfectant light and at least one of the light providing devices is adapted to emit additional light, for instance, light in the visible spectrum or infrared light. The light emitted by all light providing devices provided in the light source forms the emitted light of the light source. In an embodiment, the light source comprises a swiveling and/or adjustable light beam. For example, the light source can be a Xenon based light source comprising several light providing devices taking turns in flashing disinfectant light, wherein the flashes from the same light source comprising the several light providing devices can be provided in several different, optionally adjustable, directions. Moreover, also a 254 nm mercury UV lamp can be provided as light providing device in conjunction with adjustable reflectors or beam-shaping optics dynamically directing the light beam.

The controller can be provided as integral part of the light unit, for instance, by being provided in the same housing as the light source. However, the controller can also be provided external to the light source and being in wired or wireless communication with the light source to control the light source. In such a case the controller can be provided as part of another device, for instance, as software provided on a portable user device, like a smart phone, or as part of an overall control system for controlling more than one light unit or other devices. In this case the light unit can be regarded as comprising the light source and the controller although both are provided at different locations.

The controller is configured to control the light source, for instance, by controlling the one or more LEDs provided in the light source. In particular, the controller is adapted to control the light source such that at least a part of the emitted light is encoded with information on light characteristics of the emitted disinfectant light. The part of the emitted light that is encoded with the light characteristics can refer to the disinfectant light or can refer to an additional light provided by the light source. In a preferred embodiment, the light source is adapted to emit additional light in the infrared part of the spectrum, wherein the controller is then configured to control the light source such that the additional light is encoded with the information on the light characteristics of the emitted disinfectant light.

The information on light characteristics of the emitted disinfectant light can refer to any light characteristics that are useful for estimating a disinfection status of an object that should be disinfected with the disinfectant light. In a preferred embodiment, the light characteristics comprise information indicative of a light spectrum, intensity and/or beam characteristics of at least the part of the emitted light that is usable for disinfection purposes. The beam characteristics refer to the characteristics of the light beam provided by the light source. Preferably the beam characteristics comprise at least one of a beam size, a beam direction and a beam shape. In a preferred embodiment, the light source comprises an adjustable beam such that the beam direction, shape and size can be adjusted, for instance, by the controller. For such an embodiment it is preferred that a current adjustment, i.e. the current beam characteristics, are encoded as light characteristics in the encoded light. Based on this information the disinfection status deriving unit can, for instance, derive whether the current shape of the beam allows for a disinfection of the complete object, or only a part of the object, etc., wherein optionally for this determination the disinfection status deriving unit utilizes further information on object characteristics, like a shape, size and/or position of the object.

Additionally or alternatively, the light characteristics can also comprise information indicative of an identity of the light source, a position of the light source, for instance, mounting coordinates of the light source and/or the age of the light source in order to take a degradation of the light source into account. Further, the light characteristics can comprise information indicative of a timecode used by the light source, for instance, the information might comprise a time-stamp. Such time code information can be used by the disinfection status deriving unit to synchronize its clock, which can be useful if the disinfection status deriving unit does not have a real time clock and is not connected to any other time source. Moreover, the light characteristics can also comprise information on a time the light source has started emitting disinfectant light, a time duration of the emitting of the disinfectant light, a position of the light source, like information indicating that the light source is positioned on a ceiling, in a specific part of a room, etc. Such information when provided as light characteristics allow, for instance, an estimate of a distance between an object and the light source and/or an estimate of the time duration of an exposure of the object to the disinfectant light. For instance, information indicative of a time when the disinfectant light has been switched on and of and a current time can be encoded as light characteristics in the encoded light. The disinfection status deriving unit can then be adapted to use this information as an estimate for the duration that the disinfectant light has been provided to the object and can be adapted to further derive the disinfection status based on the estimated duration.

The controller can be adapted to encode the light of the light source that should be encoded with any known encoding method. Preferably, the controller is adapted to control the light source such that the light is encoded with a digital code. In this embodiment, the digital code is then indicative of the light characteristics. Additionally or alternatively, the controller is adapted to control the light source such that the light is encoded using a low frequency modulation or by switching the light on and off. However, also other spatial or temporal changes of the light can be used for carrying the information on the characteristics of the disinfectant light.

The sensor device comprises the detection surface and the disinfection status deriving unit. In an embodiment, the detection surface and the disinfection status deriving unit are integrated with each other, for instance, by providing the detection surface and the disinfection status deriving unit in the same housing. However, the disinfection status deriving unit can also be provided separate from the detection surface but in wired or wireless communication with the detection surface. For instance, the disinfection status deriving unit can be part of a separate device, for instance, a software running on a user device, like a smart phone, or can be part of an overall control system, controlling more than one detection surface, or controlling a plurality of different sensors, devices, and/or light units.

The detection surface is adapted to be arranged on an object. For example, the detection surface can be adapted to be placed on the surface of an object. Preferably, the detection surface is adapted to be attached to an object. For example, the detection surface can be adapted such that it can be permanently attached to an object using known attachment means, like glue, screws, etc. However, the detection surface can also be adapted to be removable attached to the object. Moreover, the detection surface can be arranged on an object by being integrated with the object, for instance, by being part of a surface of the object. Moreover, it is preferred that, if the disinfection status deriving unit is integrated with the detection surface, the arrangement of the detection surface is equal to the arrangement of the complete sensor device on the object. In particular, the attachment means can be provided as part of or attached to a housing of the sensor device.

The object on which the detection surface can be arranged can be any object. For example, the object can refer to the furniture of a room, the floor or walls of a room, the clothing of a person, a computer device, in particular, a portable computer device, like a smart phone, tablet or laptop, etc. Based on the characteristics of the object on which the detection surface should be arranged, the detection surface can be provided with different means that allow the detection surface to be arranged on the object. For example, if the detection surface should be arranged on a table, the sensor device can be provided with gluing means that allow to glue the detection surface to the table. However, if the detection surface should be arranged on a person, in particular, should be worn by a person, the detection surface can be provided with a strap that can be worn around the neck of the person. In some embodiments, the detection surface can be permanently arranged on the object. However, it is preferred that the detection surface is adapted such that it can be removable arranged on the object.

The detection surface of the sensor device is adapted to detect emitted light from the light unit, in particular, the emitted encoded light. However, the detection surface can also be adapted to detect in addition to the emitted encoded light also other parts of the emitted light of the light source that are not encoded, if present. For example, if the light source comprises a first light providing devices, e.g. LED, emitting disinfectant light which is not encoded, and a second light providing devices, e.g. LED, emitting additionally to the disinfectant light, infrared light, wherein the infrared light is encoded with the information on the light characteristics, the detection surface can be adapted to detect the encoded infrared light and also to detect the not encoded disinfectant light. Alternatively, the detection surface can also be adapted to only detect the emitted encoded light. The detection surface of the sensor device can be realized, for instance, as photo diode, photo transistor or camera device. However, the detection surface can also be realized by any semiconductor material that exhibits photo-electric properties in a suitable wavelength range. Moreover, also metals or isolators affected by an illumination in a respective wavelength range can be employed. In one example, also photovoltaic conversion cells or photoresistors can be used as detection surface of the sensor device.

The disinfection status deriving unit is adapted to derive a disinfection status of the object based on the light characteristics encoded in the emitted encoded light. The disinfection status deriving unit can be a computing device provided within a housing of the sensor device or can be regarded as software running on a computing device provided within a housing of the sensor device. However, the disinfection status deriving unit can also be regarded as a dedicated hardware. For example, the disinfection status deriving unit can be integrated in a detection surface, wherein the detection surface is adapted based on the encoded information to change one of its characteristics, for instance, its color, to indicate the disinfection status. In such an exemplary embodiment, the characteristic change of the detection surface is based on the encoded information and a fixed characteristic of the detection surface. Moreover, the disinfection status deriving unit can also be regarded as a computing device, software running on a computing device or dedicated hardware provided separate from the detection surface, but in wired or wireless communication with the detection surface to form the sensor device.

The disinfection status deriving unit is adapted to derive the disinfection status based on the light characteristics encoded in the emitted encoded light. In an embodiment, the disinfection status deriving unit is adapted to decode the information on the light characteristics encoded in the emitted encoded light and to use the decoded information for deriving the disinfection status. However, the disinfection status deriving unit can also be adapted to derive the disinfection status directly from the still encoded light being indicative of the light characteristics. The disinfection status deriving unit can derive the disinfection status of the object to which it is attached based on the light characteristics encoded in the emitted encoded light by using a functional relationship between the light characteristics and the disinfection status. For example, the functional relationship can provide a relationship between a wavelength of the disinfectant light and/or an intensity of the disinfectant light at the light source and a time necessary to achieve a predetermined disinfection status. Based on this information, the disinfection status deriving unit can then be adapted to estimate if the necessary time has already passed. However, also other functional relationships that are based on other light characteristics can be employed. Such functional relationships can be derived, for instance, from theoretical considerations on the behavior of germs when treated with light with respective light characteristics, from numerical simulations or experimental data. The functional relationship can then be provided as software to the disinfection status deriving unit or can be hardwired into the disinfection status deriving unit.

In a preferred embodiment, the disinfection status deriving unit is adapted to derive the disinfection status of the object based on the light characteristics encoded in the emitted encoded light and further based on the detected emitted light that has been detected by the detection surface of the sensor device. Preferably, the disinfection status deriving unit is adapted to derive the disinfection status based on a comparison of the light characteristics encoded in the emitted encoded light and the detected emitted light. For example, the disinfection status deriving unit can be adapted to compare an intensity, wavelength or beam characteristic of the detected emitted light with a wavelength, intensity and/or beam characteristic provided as light characteristics in the encoded light. For such an embodiment, it is preferred that the encoded light refers to at least a part of the disinfectant light such that the measurements of the disinfectant light and the light characteristics of the disinfectant light can be compared directly. However, if the encoded light does not refer to the disinfectant light, but refers to additional light provided by the light source, for instance, infrared light, the detection surface can also be adapted to detect not only the encoded light but also at least a part of the disinfectant light. Additionally or alternatively, a functional relationship between the disinfectant light and the additional light can be provided that allows the disinfection status deriving unit to derive from the measurements of the encoded light to derive light characteristics of the disinfectant light at the detection surface that can be compared to the light characteristics of the disinfectant light as provided by the light source. Based on such a comparison, the disinfection status deriving unit can be adapted to use a functional relationship between the light characteristics as provided by the light source, the light characteristics of the disinfectant light that has reached the detection surface and a disinfection status to derive the disinfection status.

The disinfection status is indicative of an expected contamination of the object with predetermined germs. The disinfection status can simply refer to a binary status, for instance, the disinfection status can refer to whether the object is disinfected or not. However, the disinfection status can also be indicative of disinfection conditions of the object between a status of being highly contaminated and being completely disinfected. Such in between conditions can be provided by the disinfection status in a continuous manner or in a quantized manner. Moreover, the disinfection status can also be germ specific, for instance, different disinfection status can be determined for different predetermined germs. Preferably, the disinfection status is determined to be indicative of a probability for disinfection indicating, for instance, that a bacterial disinfection is done for 98%. Moreover, the disinfection status might also contain or be added with a safety factor, for instance, a safety factor of 3. In such a case a signal that an object is 100% disinfected might only be provided, when a duration of exposure generally known to disinfect an object is exceeded by the safety factor.

The disinfection status derived by the disinfection status deriving unit can then be provided to a user. For example, the sensor device can provide a communication link to a user device, like a smart phone or tablet of the user, via which the disinfection status is communicated to the user device. In a preferred embodiment, the sensor device further comprises an output unit adapted to provide an output signal indicative of the derived disinfection status of the object. The output unit can refer to a display, a light output, an audio output or even to a characteristics changing surface on the sensor device. In case of a display, the sensor device can be adapted to provide the disinfection status, for instance, in form of a percentage of disinfection, wherein the value 100% can indicate that the object is completely disinfected. However, a display can also provide the disinfection status in other ways, for instance, by displaying words like “safe to be used”, “treated” “disinfected” or “contaminated”, “risk of contamination”, “do not use”. A visual output can refer to providing a light, like an LED, on the sensor device that provides a light signal when a certain disinfection status is reached. An audio signal providing unit can, for instance, provide an alarm if the disinfection status is derived to be below a predetermined threshold, like if it is estimated that the object can be contaminated above a desirable level. Moreover, if a characteristics changing surface is provided, the surface on the sensor device can, for instance, change its color or grey value based on the disinfection status.

In an embodiment, the disinfection status deriving unit is further adapted to determine a time of exposure of the object indicative of the time the object has been illuminated by the disinfectant light based on the detected light detected by the detection surface and to derive the disinfection status of the object further based on the time of exposure. For example, the detection surface can be adapted to provide a signal that indicates the time when the detection surface has first detected emitted light from the light source. This information together with information on a current time can then be used to estimate the time of exposure of the object. The disinfection status deriving unit can then be adapted to use a functional relationship between the light characteristics encoded in the emitted encoded light, the time of exposure and the disinfection status. The light characteristics can also comprise information on a location of the light source or, if known, on a distance between the object and the light source. Based on this information, the disinfection status deriving unit can be adapted to estimate an intensity of the disinfectant light on the surface of the object, and to derive the disinfection status based on the estimated intensity. The light characteristics can also comprise information on a ratio of disinfectant light and additional light emitted by the light source. The disinfection status deriving unit may then determine intensity of received disinfectant light based on measuring intensity of additional light, base on the information indicative of the ration of disinfectant light to additional light. For example, if the information encoded in the additional light indicates that the intensity of the additional light as emitted by the light source is equal (ratio of 1:1) to the intensity of the disinfectant light as emitted by the light source, the disinfection status deriving unit may measure the intensity of additional light it receives and determine that the intensity of disinfectant light received is the same.

In an embodiment, the sensor device further comprises an object characteristics providing unit adapted to provide information on characteristics of the object to which the sensor device is attached, wherein the disinfection status deriving unit is adapted to further derive the disinfection status of the object based on the object characteristics. The object characteristics providing unit that can also be regarded as the object characteristics provider is adapted to provide object characteristics of the object. The object characteristics providing unit can be a storing unit in which the object characteristics of the object at which the sensor device is arranged are stored and from which the object characteristics can be retrieved for providing the same. The object characteristics providing unit can also be a receiving unit for receiving the object characteristics and for providing the received object characteristics. For instance, the object characteristics providing unit can receive the object characteristics from a user input. However, the object characteristics providing unit can also receive the object characteristics from the object itself, for instance, if an RFID tag or a QR code marking is provided on the object storing the object characteristics. The object characteristics providing unit can be adapted to read the information from the RFID tag or the QR code and thus to receive the object characteristics. Additionally or alternatively, the object characteristics providing unit can also be adapted to communicate with a user device into which a user can input the object characteristics.

The object characteristics can refer to any characteristics that can be useful for estimating a disinfection status of the object. For example, the object characteristics can comprise information on a type of the object, for example, if the object refers to a furniture, a medical instrument, a computing device, etc. Further, the object characteristics can comprise information on where an object is placed, for instance, if an object is placed in a medical environment, a common environment, or a private environment, or where in a room an object is placed, for instance, if the object is placed in a corner of the room, in the middle of a room, etc. Additionally or alternatively, the object characteristics can also comprise information on the general structure or surface structure of an object, like whether the object comprises soft surfaces or hard surfaces, is made from metal or plastic, comprises a complex form or a form that can be compared to a box, a sphere, etc. In particular, the object characteristics can comprise information on a reflectivity and orientation in space of surfaces of the object. Further, additionally or alternatively, the object characteristics can comprise information on whether the object has been recently moved, for example, as indicated by an integrated accelerometer in the disinfection status deriving unit. For example, in an embodiment, the disinfection status deriving unit can be adapted to indicate the determined disinfection status for this object as unreliable, if the object characteristics indicate that the object has recently been moved.

The object characteristics can be taken into account by the disinfection status deriving unit for deriving the disinfection status, for instance, by taking functional relationships between the object characteristics and the disinfection status into account. For example, the disinfection status deriving unit can be provided with information how the structure of a surface impacts the disinfection process, in particular, has an influence on the time of exposure that is necessary for reaching a predetermined disinfection status. In another example, if the object characteristics indicate that the object has a form that does not allow the disinfectant light to reach all surfaces of the object, the disinfection status deriving unit can take this information into account and, for instance, expect a higher general contamination and/or a faster recontamination for this object and take these estimations into account when deriving the disinfection status. Moreover, since also the kind of surface, for instance a metal or plastic surface, can have an influence on the growth of germs such information when provided by the object characteristics can also be taken into account when deriving the disinfection status of the object.

In an embodiment, the sensor device further comprises a room characteristics providing unit adapted to provide information on characteristics of the room in which the sensor device is utilized, and wherein the disinfection status deriving unit is adapted to further derive the disinfection status of the object based on the room characteristics. The room characteristics can comprise information indicative of a need of disinfection and/or a typical contamination of the room. The room characteristics providing unit can comprise an input unit such that the room characteristics can be manually entered. Alternatively or additionally, the sensor device can comprise a reader for reading RFID tags or QR codes, wherein rooms in a facility, for instance, a hospital, are provided with the RFID tags or QR codes. Moreover, the room characteristics providing unit can also be adapted to receive the room characteristics as part of the encoded light. For example, the room characteristics can be provided as a part of a datagram transmitted by the encoded light. Since light sources are typically not moved, this information can be programmed into the light source once when the room usage is determined. If e.g. the sensor device is provided on a moveable asset, for instance, like a moveable X-ray system, even history information of different contamination and disinfection scenarios of the rooms visited by the sensor device and thus by the object to which the sensor is attached can be taken into account when determining the disinfection status of the object, for instance, the X-ray system having visited different rooms.

In an embodiment, the system further comprises a feedback providing unit adapted to provide feedback information from the sensor device to the light unit, wherein the feedback information is indicative of the disinfectant light received by the object, the disinfection status of the object and/or the object characteristics, wherein the controller is further adapted to control the light source based on the feedback information. The feedback providing unit can be part of the sensor device or can be part of the light unit or can be provided as part of both devices. For example, the feedback providing unit can be adapted to provide a communication between the sensor device and the light unit such that the sensor device can communicate the feedback information to the light unit. However, the light unit can also be provided with a feedback providing unit that is adapted to measure the feedback information, for instance, feedback information referring to the object characteristics. For example, the feedback providing unit can comprise a sensor device, like camera device, that is adapted to measure light characteristics of disinfectant light that has reached the object, object characteristics, and/or disinfection status information, for instance, disinfection status information provided by an output unit of the sensor device. In such an embodiment, the feedback providing unit is only provided as part of the light unit.

The feedback information is indicative of the disinfectant light received by the object, the disinfection status of the object and/or the object characteristics such that the controller can control the light source based on this provided information. For example, if the feedback information indicates that the disinfection status of the object is “completely disinfected”, the controller can be adapted to control the light source to switch off the disinfectant light. Moreover, also the other way around, if the feedback information indicates that the disinfection status of the object refers to “high contamination”, the controller can be adapted to switch on the disinfectant light. Further, also an intensity or duration of the disinfectant light can be controlled by the controller based on the feedback information. The controller can also be adapted to control the beam characteristics of the disinfectant light, for example, a beam size or beam direction, based on the feedback information, for instance, if the feedback information comprises information on the size, shape and/position of the object. Preferably, the controller is adapted to control the light source based on the feedback information such that it can be expected that the disinfection status of the object is increased by the measures taken by the controller. However, the controller can also be adapted to control the light source based on the feedback information such that a most effective balance between increasing the disinfection status and decreasing a power consumption of the light unit is provided.

In an embodiment, the object characteristics comprise usage information indicative of a usage of the object in a time period since a last disinfection, wherein the usage information is provided such that from the usage information an expectable contamination can be estimated. The usage information can refer, for instance, to an estimate how many times the object has been touched by a person in a time period since a last disinfection or an estimate how long the object might have been in contact during its usage with contaminated surfaces. For example, if the object is a computer device comprising an input unit like a smart phone display, the usage information can refer to the number of registered touches of a finger on the display. Since it can be expected that an object that is used more often in a time period since the last disinfection, comprises a higher contamination with germs than an object that only has been used seldom or not at all in the time period since the last disinfection, this usage information can be taken into account by the disinfection status deriving unit when deriving the disinfection status and can also be taken into account as part of the feedback information by the controller for controlling the light unit. For example, after the same exposure time the disinfection status deriving unit can be adapted to derive a lower disinfection status for an object that has been used more often than a comparable object that has been used seldom or not at all. Moreover, if the feedback information indicates that an object has been used more often than other comparable objects, the controller can be adapted to provide the disinfectant light over a longer time period, with a higher intensity or with a different wavelength to this object compared to light provided to other comparable objects that have not been used so often.

In an embodiment, the usage information can also include smartphone based anonymous contact tracing information. For instance, it can be determined based on a contact tracing app that a person sitting at a desk yesterday has today been tested positive for Corona virus. The object and/or the sensor device arranged on the object can then be informed by the contact tracing app and this information can be provided as part of the usage information to the lighting unit and/or the disinfection status deriving unit. The controller can then be adapted to control the light unit such that the disinfectant light is provided such that in particular the objects in contact with the person are disinfected, in particular, such that also viruses are deactivated. Moreover, also the disinfection status deriving unit can use this information directly and set the disinfection status directly to “contaminated” until the object is disinfected with the disinfectant light.

In an additional or alternative embodiment, the usage information can also include recent tasks executed by the person. For example, if a person in a commercial kitchen has recently cut raw chicken meat, a surface on which the meat has been cut is more likely to be contaminated with salmonella. Moreover, the usage information can comprise information on whether a person using the object has recently performed on his/her body a decontamination, for example, using a free-standing disinfection portal placed at an entrance of a shared space to disinfect employees upon entering. The disinfectant status deriving unit and the controller of the lighting source can then be adapted to take this information into account.

In an embodiment, the object characteristics comprise information on a reflectivity and orientation of object surfaces and are part of the feedback information. The controller can then be adapted to take this information into account to model how much of the disinfectant light is reflected by the object being, for instance, a metal table, to other areas of the room. The controller can then control the light source in accordance to this model, for instance, by providing the light beam of the light source in a direction that allows to use reflections of the disinfectant light on reflecting surfaces of the object in order to disinfect hard-to-reach areas outside of the direct field of view of the light source. Moreover, the object may also have several different settings for its orientation, which the end-user can adjust. For instance, a table surface may be adjustable in height or tilt. The information on such an adjustment can also be part of the object characteristics such that the model can be adjusted accordingly. Moreover, the controller can be adapted to provide a user with information on how to adjust the object to archive a predetermined disinfection goal. Similarly, if disinfectant lighting is used in a factory, the status of a machinery may influence the disinfection. For instance, in a first state the large metal lid in a commercial kitchen may be open, in a second stage the metal lid may be closed.

In an embodiment, the system, for example, the lighting unit and/or the sensor device, comprise clutter or soil sensor means adapted to deduce a clutter or soil status, e.g. whether a to-be-disinfected surface of the object is presently cluttered with objects, soiled and/or dust covered, wherein the disinfection status deriving unit can further base the deriving of a disinfection status on the clutter or soil status of the object. For instance, a kitchen table may be at times cluttered with plates and cutlery, wherein the clutter may be detected by various means such as thermopile sensors, radio frequency sensors or a low resolution camera being utilized as clutter sensor means. If the clutter or soil sensor is provided as part of the light unit, the controller can be adapted to provide information about a clutter or soil status of the object surface via the encoded light to the disinfection status deriving unit. The disinfection status deriving unit can then use this information, for instance, to determine a confidence level of the disinfection status of the object. Moreover, the light unit can also provide information about the soil status, e.g. a soil layer on the object, via the encoded light to the disinfection status deriving unit, wherein the disinfection status deriving unit can be adapted to determine the disinfection status further based on this information. For example, it is known that surfaces with a thick soil layer may pre-absorb the UV-C photons before they reach an active virus or bacterium. This effect can thus be taken into account by the disinfection status deriving unit when deriving the disinfection status. In a preferred embodiment, a time since the last standard manual cleaning of an object surface, for instance, by a nurse, can be determined and taken into account to determine the effectiveness of the disinfectant light on the surface.

In an embodiment, the sensor device further comprises a biosensor unit adapted to detect biological components contaminating the object, and wherein the object characteristics comprise information indicative of a detection result of the biosensor. The biosensor can be, for instance, a fluidic microsensor that is adapted to analyze, for example, an air contamination. Such a sensor can use, for example, analysis techniques similar to point of care devices, like point of care blood analysis devices used for diabetes or infectious diseases. The biological components can refer to bacteria, viruses and/or fungi that can contaminate an object. The result of the detection of the biological components can, for instance, be used for estimating a contamination of the object. The disinfection status deriving unit can then further take the result of the biosensor into account for deriving the disinfection status of the object. Additionally or alternatively, the result of the biosensor can be part of the feedback information and the controller of the light unit can be adapted to control the light source based on the result of the biosensor.

In an embodiment, the controller is further adapted to determine based on the feedback information a measure for the effectiveness of the disinfection and to control the light source based on the determined effectiveness measure. The effectiveness measure is indicative of the effectiveness of the disinfection of the object. For instance, the effectiveness measure can be indicative of an increase of the disinfection status during a predetermined time period or can be indicative of an increase of the disinfection status when applying a certain light intensity or wavelength. Based on the effectiveness measure, the controller can then be adapted, for instance, to decide if a change in the light characteristics of the disinfectant light is necessary, for instance, to increase the effectiveness of the disinfection. Moreover, if the effectiveness measure indicates that a current disinfectant light setting is not effective enough, for instance, to reach a predetermined disinfection goal, the controller can also be adapted to assign a different light source to the disinfection of the object. For example, if the effectiveness measure indicates that light of a first light source of the light unit is obstructed in its field of view, the controller can be adapted to control another light source or even another light unit to provide disinfectant light to the object that might be more suitable for disinfecting the object.

In an embodiment, the light source is adapted to emit light with a wavelength in the ultraviolet spectrum, in the infrared part of the spectrum or in the purple part of the visible spectrum as disinfectant light, and wherein the light source is controlled by the controller such that the disinfectant light is encoded, and/or wherein the light source further emits light with a wavelength below the ultraviolet spectrum in addition to the disinfectant light, wherein the light source is controlled by the controller such that the additional light is encoded.

In an embodiment, the system further comprises a motion sensor, wherein the motion sensor is adapted to detect the presents of a living being in the space lighted by the light source and the controller is adapted to control the light source such that disinfectant light is only provided when no presents of a living being is detected in the space. This embodiment has the advantage that a health risk provided by the used disinfectant light can be avoided, even in cases in which UV-C light is used as disinfectant light.

In an embodiment, the light source is adapted to emit disinfectant light only into a predetermined direction with respect to a fixation means of the light unit. For example, the direction can be determined such that, if the light unit is fixed in accordance with an intended fixation of the fixation means in a room, the light source emits the disinfectant light only in an upper direction, and thus an upper part of the room, preferably in a direction above a horizontal direction. In such an embodiment, objects provided above the light unit, for instance, at a ceiling of a room, can be disinfected without applying the disinfectant light to lower objects and surfaces, for instance, without applying the disinfectant light to occupants of the room. For example, even if the surface is out of reach of humans, viruses can attach and detach from the surface and reach a human. In such an embodiment for disinfecting upper surfaces, a wavelength range can be chosen, which is not acceptable at lower heights in the room where it can reach living beings. In practice, such an embodiment is advantageous in rooms with high ceilings. Preferably, the direction of the emitted light is such that, if the light unit is fixed in a room in accordance with a provided specification only the air above 2.1 meters is irradiated with the disinfectant light.

In another aspect of the invention, a light unit is presented, wherein the light unit comprises a) a light source adapted to emit light, wherein at least a part of the emitted light is disinfectant light usable for disinfection purposes, and b) a controller configured to control the light source, wherein the controller is configured to control the light source such that at least a part of the emitted light is encoded with information on light characteristics of the emitted disinfectant light.

In another aspect of the invention, a sensor device is presented, wherein the sensor device comprises a) a detection surface adapted to be arranged on an object, and to detect emitted light from a light unit as described above, wherein the detection surface is adapted to detect at least the emitted encoded light, and b) a disinfection status deriving unit adapted to derive a disinfection status of the object based on the light characteristics encoded in the emitted encoded light.

In another aspect of the invention, a method for disinfection applications is presented, wherein the method comprises the steps of a) providing a light source adapted to emit light, wherein at least a part of the emitted light is disinfectant light usable for disinfection purposes, b) controlling the light source such that at least a part of the emitted light is encoded with information on light characteristics of the emitted disinfectant light, c) arranging a detection surface on an object, d) detecting by using the detection surface the emitted encoded light from the light source, and e) deriving a disinfection status of the object based on the light characteristics encoded in the emitted encoded light.

It shall be understood that the system, the light unit, the sensor device and the method have similar and/or identical preferred embodiments, in particular, as defined in the dependent claims.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily an embodiment of a system for disinfection applications comprising a light unit and a sensor device,

FIG. 2 shows a flowchart exemplarily illustrating an embodiment of a method for disinfection applications, and

FIGS. 3 and 4 illustrate exemplary applications of the system and the method.

### DETAILED DESCRIPTION OF EMBODIMENTS

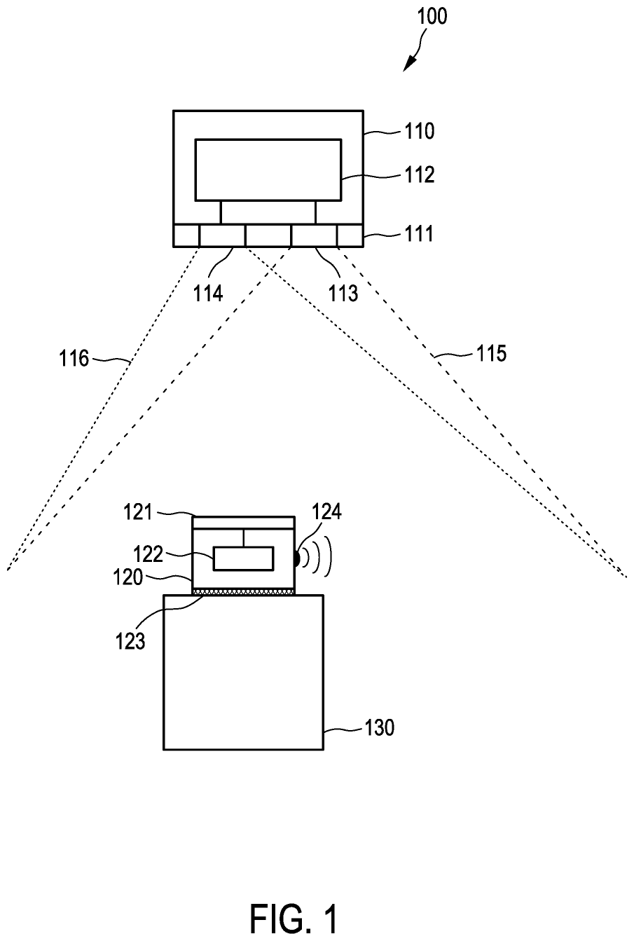

FIG. 1 shows schematically and exemplarily an embodiment of a system for disinfection applications comprising a light unit and a sensor device. In this exemplary embodiment, the system 100 comprises the light unit 110 and the sensor device 120, wherein the sensor device is attached to an object 130 with attachment means 123 optionally provided with the sensor device 120. In this example, the attachment means 123 refer to a glue that glues the sensor device 120 to the surface of the object 130. However, in other embodiments the attachment means 123 can refer to completely different attachment means like screws, straps, a hook and loop fastener, magnets, etc. Moreover, the attachment means 123 can also be omitted and/or the sensor device 120 can be an integral part of the object 130.

The light unit 110 is provided somewhere in the vicinity of the object 130 such that light emitted by the light unit 110 can reach the object 130. Preferably, the light unit 110 is attached to a ceiling of a room in which object 130 is placed. The light unit 110 comprises a light source 111, wherein the light source 111 in this example comprises as light providing devices a first LED 113 and a second LED 114. The first LED 113 is adapted to emit light that is usable for disinfection purposes. Preferably, the disinfectant light 115 comprises light with a wavelength of 405 nm. However, the disinfectant light 115 can also comprise light with smaller wavelengths, for instance, light in the ultraviolet part of the electromagnetic spectrum, preferably, UV-C light, or light with longer wavelength, for instance, infrared light, for specific applications. The second LED 114 is in this exemplary embodiment adapted to emit visible light 116 that can be used for lighting purposes, for instance, for lighting at least part of a room with human inhabitants. The disinfectant light 115 emitted by the first LED 113 and the additional light 116 emitted by the second LED 114 together form the emitted light emitted by the light source 111.

Light unit 110 further comprises a controller 112 that is adapted to control the light source 111, in particular, by controlling the first LED 113 and the second LED 114. For instance, the controller 112 can be adapted to control a switching on and a switching off of the first and the second LEDs 113, 114. However, the controller 112 can also be adapted to control the light characteristics of the first LED 113 and the second LED 114, for instance, a spectrum, a light intensity, a beam characteristic, like a beam size, etc. of the light emitted by the first LED 113 and the second LED 114. Moreover, the controller 112 is adapted to control at least one of the first LED 113 and the second LED 114 such that the light emitted by this LED is encoded with information on the light characteristics of the emitted disinfectant light 115. For example, the controller 112 can be adapted to control at least one of the first LED 113 and the second LED 114 by applying a low frequency modulation or a fast on and off switching to the LED 113 or 114 that is not visible to the human eye. In a preferred embodiment, the additional light 116 provided by the second LED 114 is encoded with the information on the light characteristics of the disinfectant light 115. However, also the disinfectant light 115 provided by the first LED 113 can be encoded with the information on the light characteristics. Moreover, in an embodiment, even both the additional light 116 and the disinfectant light 115 can be encoded with the light characteristics information.

The sensor device 120 arranged on object 130 comprises a detection surface 121. The detection surface 121 can refer, for instance, to a photo detector or any other known detector unit that is adapted to detect light in the desired part of the spectrum. The detection surface 121 is adapted to detect at least the light emitted by the light unit 110 that is encoded with the light characteristics. For example, if the additional light 116 provided by the second LED 114 is encoded with the light characteristics, the detection surface 121 is adapted to detect at least this additional light 116. If in another example the disinfectant light 115 provided by the first LED 113 is encoded with the light characteristics, the detection surface is adapted to detect at least the disinfectant light. Preferably, the detection surface 121 is adapted to detect light in a wide spectral bandwidth such that it can detect the additional light 116 and the disinfectant light 115 concurrently.

Further, the sensor device 120 comprises a disinfection status deriving unit 122, wherein the disinfection status deriving unit 122 is adapted to derive a disinfection status of the object 130 based on the light characteristics encoded in the light of the light source 111, for instance, in the additional light 116. For instance, the disinfection status deriving unit 122 can decode the light characteristics information based on the detection results of the detection surface 121. The decoded light characteristics information can then be used to derive, in particular estimate, a disinfection status of the object 130. However, the decoding of the light characteristics can in some cases also be omitted and the detection result of the detection surface 121 can directly be used for deriving the disinfection status of the object 130. In particular, in such a case the disinfection status deriving unit 122 is adapted to utilize the light characteristics information in its still encoded form for deriving the disinfection status.

In an example, the disinfection status deriving unit 122 is adapted to derive a disinfection status of the object 130 by using a functional relationship between the light characteristics of the emitted disinfectant light 115 and a disinfection status. For example, light characteristics information comprising the emitted wavelength and intensity of the disinfectant light 115 at the first LED 113 allows an estimation of the disinfection status of the object 130. Functional relations between light characteristics and a disinfection status can be determined, for instance, by experiments, calibration measurements, and even by theoretical simulations including the reaction of different germs to disinfectant light with different light characteristics. Moreover, the functional relationships can also be provided as simple rules based on experience or on measurements. Such a rule can, for instance, simply state that the germs on a surface will have been reduced to substantially zero, if disinfectant light 115 of the respective wavelength provided by the first LED 113 has been provided to the object over at least a specific time, wherein in this case a safety factor might be provided to the time, since such a simple rule might only provide a rough estimate. However, also more complex rules or functional relations can be utilized for deriving a disinfection status of an object.

To increase the accuracy of the derived disinfection status, the disinfection status deriving unit 122 can additionally also be adapted to take into account light characteristics of the light detected by the detection surface 121, like wavelength, beam size, intensity, etc., for deriving the disinfection status. For example, a measurement of the light intensity of the light received by the detection surface 121 compared with the light intensity of the emitted light at the light source 111 as encoded as part of the light characteristics in the emitted light allows to estimate a distance between the object 130 and the light unit 110, wherein the distance can also be part of the functional relationship between the light characteristics of the disinfectant light 115 and the detection status.

Additionally or alternatively, also other information can be taken into account to increase the accuracy of the derived disinfection status. For example, also information on object characteristics of the object 130 can be taken into account. The functional relationship can in this embodiment, for example, also depend on information on a surface of the object 130 provided as object characteristics. For instance, the growth of germs on metal surfaces can be determined to be very different from the growth of germs on plastic or wood surfaces, wherein different growth rates on these surfaces can then be taken into account in the functional relationship utilized for determining the disinfection status. Another alternative or additional example refers to use information that is indicative of a usage of the object 130. For example, in such a case the functional relationship can also be dependent on a frequency or length of physical contact with a user, wherein for a higher contact rate a higher contamination can be expected.

The derived disinfection status of the object 130 can then be provided as information to a user using, for instance, an output unit 124. The output unit 124 can refer to a transmitter that is adapted to transmit the information to a user device, for instance, a smart phone, wherein the disinfection status can then be provided by the user on the display of the user smart phone. However, the output unit 124 can also refer to an output unit providing a visible or audible signal like a light signal or an alarm signal that are indicative of the disinfection status. For example, the output unit 124 can provide a green light when the disinfection status of the object 130 has reached a predetermined threshold, in particular, when it can be expected that the object 130 can be used safely, i.e. with only a very low chance of infection, by a user. If such a safe disinfection status is not yet reached by the object 130, the output unit 124 can provide a red light. In this case, the user can directly be provided with the information if the object 130 is safe to use or not. However, the output unit 124 can also be adapted to provide an alarm, in particular, in a case in which the object 130 reaches a disinfection status that is not safe to use anymore. In other embodiments, the output unit 124 can also refer to a display for displaying the disinfection status, to an information surface which changes at least one characteristic being indicative of the disinfection status, for instance, the information surface can change its color or grey value based on the disinfection status.

Moreover, the output unit 124 can also be a transmitter that is adapted to transmit the disinfection status as feedback information to the light unit 110, wherein in this case the light unit 110 is preferably adapted such that it can receive the feedback information from the sensor device 120. The controller 112 can in this case use the feedback information of the sensor device 120, for instance, the information on the disinfection status, to control the light source 111, in particular, the first LED 113 providing the disinfectant light 115.

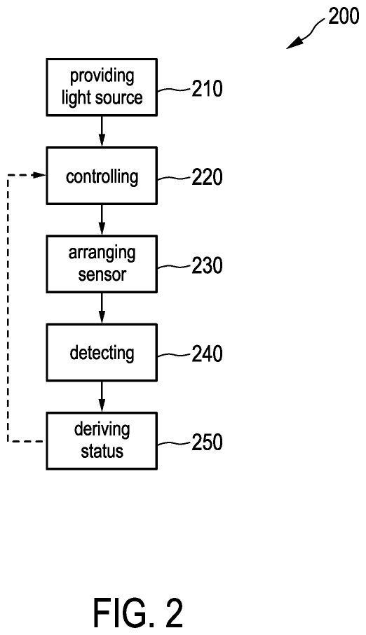

FIG. 2 shows exemplarily a flowchart of a method for disinfection applications. The method 200 comprises a first step 210 of providing a light source adapted to emit light, wherein at least a part of the emitted light is disinfectant light usable for disinfection purposes. For example, a light source like the light source 111 as described above can be provided. Further, the method 200 comprises a step 220 of controlling the light source such that at least a part of the emitted light is encoded with information on light characteristics of the emitted disinfectant light. For example, the step 220 of controlling the light source can be carried out by a controller 112 in a manner exemplarily described above. In step 230, further a sensor device is arranged on an object, wherein an example of the sensor device is the sensor device 120 described above that can be attached to the object 130 in accordance with the above explanations. Further, the method 200 comprises a step 240 of detecting the emitted encoded light from the light source using, for instance, the detection surface 121 of the sensor device 120. In a final step 250, a disinfection status of the object is derived based on the light characteristics encoded in the emitted encoded light using, for instance, the disinfection status deriving unit 122 as described above. Optionally, as indicated by the dashed line, the derived disinfection status can be provided as part of feedback information that can be taken into account during the controlling step 220. In this embodiment, in step 220 the disinfection status can be taken into account when controlling the light source such that the disinfection status of the object is increased with respect to a present disinfection status. In this embodiment, after having received the feedback information and taken the feedback information into account in step 220, the next step 230 of arranging the sensor device can be omitted, if the position of the sensor device should be kept fixed. However, based on the feedback information the sensor device can also be rearranged in step 230.

In the following, some preferred embodiments and applications of the system for disinfection applications are described in more detail.

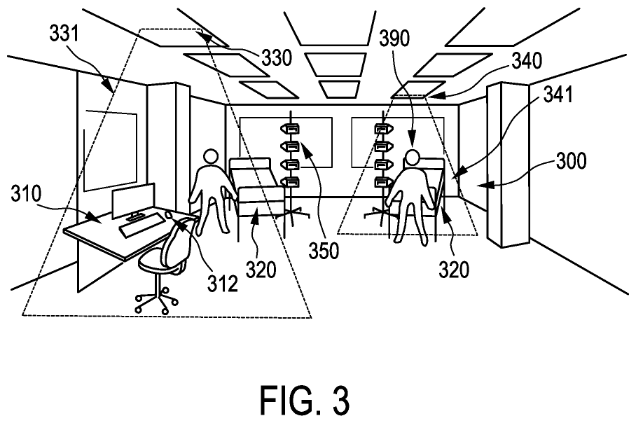

Depicted in FIG. 3 is a typical intensity care unit 300 situation where care givers 390 have special medical workplaces 310 comprising computer equipment 312 which are near to beds 320. In addition, there are moveable appliances 350, for instance, infusion towers, respiration appliances or heart monitors, which can be brought to the position where they are needed. An illumination is typically recessed in the suspended ceiling. Light units 330 and 340 referring, for instance, to a light unit as described with respect to FIG. 1, can be provided additionally or alternatively to normal illumination devices in the room and are adapted to emit disinfectant light 331, 341 like mentioned above. In an embodiment, the light beams emitted by the light units are carrying a digital code indicative of light characteristics of the disinfectant light. The digital code can be provided, for instance, by a low frequency modulation, utilizing a yellow dot coding, or utilizing LiFi. These light characteristics can contain among other information in particular information on an intensity and wavelength distribution of the disinfectant light.

In an embodiment, the light characteristics can comprise the wavelength of the disinfectant light emitted together with information on an intensity and beam sizes over distance. With this information appliances and workplace machineries comprising a sensor device as described above can deduce the grade of disinfection, i.e. a disinfection status, when keeping track of the time of exposure. Local signaling may show that sufficient exposure has been accumulated for safe continuous usage intermittent to the conventional cleaning.

Different kinds of equipment may have special disinfection rules like, e.g., the computer mouse 312, the keyboard or the user interface devices of an appliance. The disinfection rules may combine special chemical means with optical means. Such information can be part of the object characteristics information and can be taken into account by the disinfection status deriving unit when deriving a disinfection status. For example, a basic dose calculation for calculating a received radiation dose can be based on a functional relationship for a flux intensity. A spectral content can be calculated from the light characteristics encoded in the emitted encoded light comprising information on the spectral composition. Moreover, also a special sensitivity curve of the detector surface of the sensor can be taken into account for deriving the disinfection status. This can be beneficial, for example, when a surface finish of the sensor, for instance, a surface structure, color, etc., acts as an artificial filter.

Since a form factor and height of a room as well as positions of furniture and equipment can play an important role for the disinfectant lighting reaching the target area, it is desired to automate the feedback loop instead of a lighting designer calculating once the right emitted light dosage per fixture. In addition, the wall-paint of rooms can also play a role and might change over time. Thus, providing feedback information, for instance, as described above, to the light unit and controlling the light source based on the feedback information allows to adapt the disinfectant light also to changes in the environment of the light unit.

In an embodiment, the sensor device or light unit can receive information on a grade of contamination as part of the object characteristics. For example, information provided by a computer mouse or a keyboard device about periods of usage can be utilized as indication to roughly estimate contamination. Similarly, context knowledge about how frequently a wall switch has been touched may be used to calculate the disinfection dose required to inhibit the growth of mold, mildew, fungi and other odor-causing bacteria between normal cleanings in an elderly home. Some information on periods of use can increase an expected contamination and periods of lighting disinfection exposure can then decrease it.

While purple light disinfection shows first effects after 90 min, only after a few weeks it reaches highest level of disinfection by suppression of germ growth in between the regularly scheduled conventional cleaning. Hence, if the disinfection cleaning has only been active for two days, the level of germs is still relatively high. Thus, in exemplary embodiments, for different spectral components the effectivity for these may also be part of a functional relationship used for deriving the disinfections status when the spectral content of the disinfectant light is provided as light characteristics in the emitted encoded light.

The sensor device can also feature a biosensor measuring, for instance, the local germ content. Also the results of the measurements can be used to derive the disinfection status. Additionally, if more than one biosensor is provided by the sensor device or if more than one sensor device is provided in a defined area, like a room, the results of the measurements of all these biosensors can be taken into account for deriving the disinfection status, for instance, by modelling how germs are moved through the defined area, e.g. from a first sensor to a second sensor, depending on the chosen disinfectant lighting.

An especially advantageous application of the above described system is to utilize the system to clean continuously those areas where harmful bacteria are known to enter a facility such as a waiting room in a doctor's office or hospital and in this way to prevent the spread of germs across the entire facility, wherein the sensor device can provide a disinfection status of often used objects like chairs or tables and thus indicate if an additional cleaning, for instance, by hand, is necessary.

In a preferred embodiment, the controller can be adapted to remotely turn the disinfectant light on or off, for instance, based on feedback information containing a disinfection status information. Moreover, the controller can also be adapted to continually regulate the disinfectant light intensity. For example, information on a presence of living beings can be provided to the controller, for example, by a light switch in communication with the light unit. This information can then be used by the controller for controlling an intensity and/or a spectral composition of the disinfectant light. Moreover, information on a power efficiency, variable electricity rates and/or demands can also be taken into account as optimization criteria by the controller for controlling the disinfectant light, for instance, when there is sufficient time for the exposure. Not so efficient exposure may be selected when the contamination is expected to be low.

In an embodiment, the sensor device, in particular, the disinfection status deriving unit, can be adapted to determine a contribution for different light units from which the light reaches the sensor device. In particular, the disinfection status deriving unit can be adapted to differentiate between the different information, for instance, different light characteristics, encoded in the light emitted by the different light units. Information on the contribution of different light units can also be part of the feedback information and can be provided to the controller of the light units. The controller of the light units can then be adapted to control the light units based on this information, for instance, by switching off light units with only a low impact on a contaminated area.

In an embodiment, the sensor device can be adapted to comprise a dose check functionality. For example, the sensor device can be provided with information on a highest allowable disinfectant light dose, for instance, of the highest allowable dose of UV-C light, in a given time, for example, during a work week, wherein the dose check functionality can then provide a user with information whether he/she is allowed to be exposed to the disinfectant light or whether he/she should better avoid the disinfectant light. The highest dose information in such an embodiment might be provided in form of hardware reacting to a predetermined amount of the disinfectant light. However, the information can also be stored on a storage such that, for instance, the disinfection status deriving unit can access the information for determining if the maximal dose is already reached.

In an embodiment, the disinfection status derived by the disinfection status deriving unit can be provided by local signals, for instance, indicating a status of a contamination taking into account a recent usage, intermittent cleaning, and/or light disinfection. For example, a red light can be provided for high contamination, a yellow light for medium and a green light for low contamination. Such signaling may also be provided on a room or building level. For example, one or more sensor devices can communicate their derived disinfection status to a common controller, wherein the common controller can then be adapted to derive an overall disinfection status of an area from the provided disinfection status and to control, for instance, the light units to provide visible light indicative of the overall disinfection status, or a dedicated output unit to inform a user of the overall disinfection status. Such an overall disinfection status can then be used, for example, in order to select a room for the next patient or recommend certain toilets in an office utilizing the room-booking space-management apps commonly available to employees of high-tech companies.

Some research results suggest that disinfectant light in combination with disinfectant chemicals is more effective. Hence, the disinfection status provided by the sensor device can also be used in order to schedule disinfectant application. This application can be supported by providing information on the kind of used disinfectant to the disinfection status deriving unit, wherein the disinfection status deriving unit can be adapted to further derive the disinfection status based on this information. Near field communication (NFC) or QR code tags on the container for an applied disinfection chemical may be another technique especially for furniture to learn about a disinfectant applied. The sensor device can in this case be provided with reading means for such tags. After applying a disinfectant, the sensor device may be placed in a position to the container to read the related information and may adjust the disinfection status determination appropriately.

In an embodiment, the sensor device can comprise photo voltaic (PV) cells to supply themselves with power. In order to support this the spectrum of the light emitted by the light source may be adapted to provide light for easy PV power conversion, e.g. by providing additional infrared light. For example, the feedback information can contain information on the power status of the sensor device and/or the light spectrum usable by the PV of the sensor device. The controller of the light unit can then be adapted to use this information to control the additional light, for instance, to activate the additional light when power is required by the sensor device. The sensor device can be added to commercially available solar-powered multi-sensors comprising, for instance, an accelerometer, magnetic contact detector, temperature and/or humidity sensors that can be mounted on surfaces and chairs.

In an embodiment, at least the detection surface of the sensor device can be adapted to be carried by a user, for instance, on the clothing, around the neck or around the wrist. The sensor device can then be adapted to determine accumulated low wavelength light exposure like a dosimeter. Further, the sensor device can be adapted to warn the user when excessive exposure was found. The sensor device can be adapted to determine the photobiological safety considerations regarding thermal, retinal and skin damage following IEC standards.

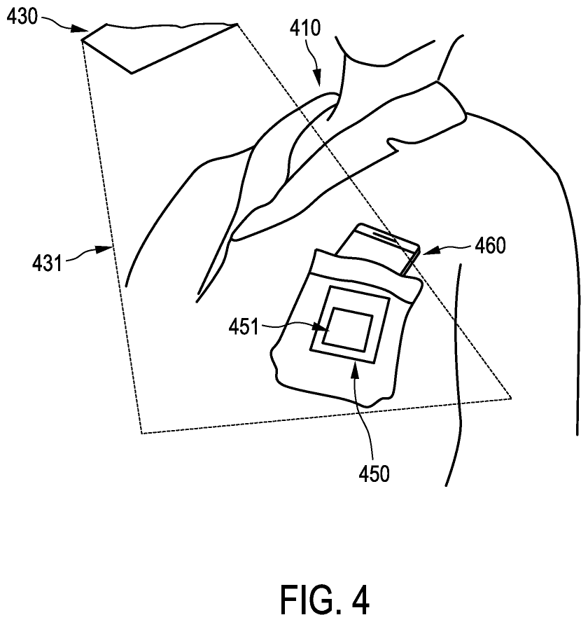

FIG. 4 shows an example of how a detection surface with a dosimeter functionality for disinfectant light could look like. In such an example, the detection surface 450 should be exposed to the light beam 431 as emitted by the light unit 430. In FIG. 4 the detection surface 450 is provided in form of a sticker at the breast pocket of a person 410 to be protected. Further, an output unit is provided in order to visually inform about the disinfection status, e.g. by means of a display 451 or signaling lights. Moreover, at least part of a sensor device comprising a dosimetric functionality can be integrated with a moveable communication appliance like a smart phone 460, as the disinfectant light is in the visible light spectrum.

In an embodiment, a user device, like a smart phone and/or point of care appliance, can be provided with the disinfection status information derived by the disinfection status deriving unit and can thus receive information indicative of the contamination load, e.g. related to the room or the health status of people in the vicinity.

In an embodiment, the light unit can further comprise a sensor which measures reflectance of part of the disinfectant light, for instance, of UV or purple light or other components active in the disinfection, as feedback information. The controller can then be adapted to determine whether an object is beneath it and for how long it remains there and control the disinfectant light accordingly. When the object has been disinfected, additional visible light provided by the light unit can indicate this, e.g. by a change in color.

Moreover, such a sensor can further be utilized to determine the reflectivity level and/or distance of the radiated object and the controller can be adapted to determine an effectiveness of the radiation provided to the object. The controller can then control the light source based on this effectiveness, for instance, by controlling the size of the disinfectant light beam. Moreover, the information can also be encoded in the light provided by the light source such that the sensor device can receive the information and derive the disinfection status accordingly. In an example, the sensor can be utilized to analyze the reflectivity of the disinfectant light by a wall of the room. This can then be taken into account when controlling the light source, for instance, to provide disinfection to areas not directly illuminated by the light unit, in which case reflections of walls and objects are crucial.

In an application, feedback information provided by the sensor device can be utilized to control the light source in complicated room settings. For example, the sensor device can be used to determine the disinfectant light reaching different areas of a room, for instance, a floor or a countertop in a commercial kitchen. Generally, it is expected that a floor of a room requires a higher dose as it is further away from the disinfectant light source mounted within a ceiling, as well as since the floor is hard to reach because of much shadowing caused by objects. The detection surface can then be arranged in the corners of the floor hardest to reach and the disinfectant light can be controlled based on the feedback information from the sensor device.

In an embodiment, the sensor device can also be used to estimate the effectiveness of the disinfection in an air column between the light unit and the detection surface, since light-based disinfection is known to also kill air borne germs.

In an embodiment, the sensor device can also be used to monitor cleaning efficiency in certain areas hard to clean with conventional cleaning. For instance, disinfectant lighting can kill harmful bacteria also on soft surfaces, e.g. of a bed blanket, couch, while conventional cleaning only works on hard surfaces. Hence, monitoring the effectiveness of the disinfectant lighting on the soft surfaces can be of more importance than on the hard surfaces.

Moreover, the sensor device can also be used to recognize if a semi-transparent object is inserted between the light unit and the object. For example, the disinfection status deriving unit can be adapted to determine based on the light detected by the detection surface a change in the characteristics of the light detected by the sensor device indicating a change between the detection surface and the light unit. Information on such changes can allow, when provided as feedback information, for instance, the controller to determine if disinfectant light is scattered in a non-intended direction. Moreover, also feedback information of a second sensor device can be utilized to determine scattered light.

Although in above embodiments the light source was described as comprising two light providing devices, e.g. LEDs, in particular, a first LED providing the disinfectant light and a second LED providing additional light, in other embodiments only one light providing device, e.g. LED, can be provided that can be adapted to provide only the disinfectant light or that can be adapted to provide the disinfectant light and additional light. Moreover, in other embodiments, more than two light providing devices, e.g. LEDs can be provided, for instance, a plurality of light providing devices, e.g. LEDs can be provided that can provide disinfectant light, for instance, of the same or of different wavelengths, and also more than one light providing device, e.g. LED, can be provided that provide additional light of the same or different parts of the electromagnetic spectrum. In a preferred example, at least one light providing device, e.g. LED, is provided that can emit disinfectant light and also light that is suitable for lighting a room and additionally one or more light providing devices, e.g. LEDs, are provided that emit infrared light, wherein in this embodiment it is preferred that the controller is adapted to control at least the infrared light providing devices such that the light characteristics of the disinfectant light are encoded in the infrared light.

Although in the above embodiments the light source was described as comprising as light providing devices LEDs, in other embodiments also other light sources can be utilized. In particular, the disinfectant light can be generated by, for instance, low-pressure mercury germicidal lamps, pulsed xenon arc germicidal ultraviolet irradiation lamps, or rare gas-halogen, for instance, krypton-chlorine, discharge light sources. Generally, the realization of the light source and the utilized wavelength can be chosen based on the specific application of the system for disinfection applications, for example, whether the system should be applied in an environment in which living beings might be subjected to the disinfectant light or not. Some general guidelines for the choice of the wavelength and some preferred embodiments will be given in the following.

In an embodiment, germicidal ultraviolet light (GUV) referring to short-wavelength ultraviolet “light”, i.e. radiant energy, that has been shown to kill bacteria and spores and to inactivate viruses, can be used. In particular, GUV light with wavelengths in the photobiological ultraviolet spectral band, known as the “UV-C”, from 200 to 280 nm, have shown to be the most effective for disinfection, although longer, less energetic UV light, for instance, UV-A, can also disinfect if applied in much greater doses. While UV-A and longer, for instance, visible wavelengths do not have germicidally effective emission wavelengths to inactivate viruses, they have been proven to effectively fight microbes. Although UV-A light and light in the visible spectrum have shown to be less effective than UV-C light, in some applications the usage of UV-C light or even UV-A light might not be acceptable, for example, due to material damage by the UV light or health risks to living beings. Thus in such applications it is preferred that the light source is adapted to provide UV-A or violet visible light, for instance, with a wavelength of 405 nm. In such applications, the light doses provided to the object can be such that a viral sterilization can also be ensured with these light sources. LEDs as light providing devices in these embodiments have the advantage that they can easily be incorporated into LED-based luminaires, and that the dose may be chosen such that for an 8-hour daily exposure no protective gear is needed. In embodiments in which germicidal ultraviolet irradiation (UVGI) should be utilized, for instance, in the UV-C wavelength region, the UVGI light can be provided by many different lamp technologies. For example, low- and medium-pressure mercury UVGI lamps emitting UV light that has proven to kill both viruses and bacteria can be used as light providing devices. Also LED devices can be used that emit light with a wavelength near 270 nm as light providing devices. Moreover, also “Far UV-C” lamps that emit light with a wavelength around 222 nm can be used as light providing devices. Such light sources have the advantage that they are safe for the eyes and skin of living beings, in contrast to light with longer wavelength UV-C, for instance, emitted by mercury lamps. Also pulsed xenon arc UVGI lamps emitting UV and visible radiant energy that has proven to kill both viruses and bacteria can be used as light providing devices. In particular, in embodiments in which no living beings are exposed to the disinfectant light these light sources can be applied. In some embodiments the pulsed xenon arc lamps can be filtered so that only the UV light used for disinfection is emitted. In a further alternative embodiment, rare gas-halogen discharge light sources can be used that produce a significant emission in the Far UV-C region, i.e. in a wavelength range from 205 to 230 nm, as light providing devices. An advantage of these light providing devices is that the deactivation rate of some bacteria and viruses is relatively high, and the effect of the emission on human skin and eyes is much reduced compared to the light at a wavelength of 253.7 nm. Generally, Far UV-C light sources are preferred in many applications, since they allow for an efficient and safe continuous disinfection in the presence of humans. For example, it is well established that UV-C light (231-280 nm) must be shielded from humans as it poses a carcinogenic safety risk, whereas at the Far UV-C range (200-230 nm), it has been shown that the risk is strongly reduced, since the Far UV-C light neither penetrates the top layer of the human skin nor the tear layer of the eye. Thus, while Far UV-C light cannot reach or damage living human cells, it can still penetrate and kill the very small viruses and bacteria floating in the air as well as disinfect office tables and other surfaces.

Therefore, in applications of the system for disinfecting surfaces in potentially occupied building spaces, a light source being adapted to emit light in the Far UV-C spectrum is preferred. However, in applications in which it can be guaranteed that nobody is present in the space during the application of the UV light, also light sources adapted to emit UV-C light, like, mercury lamps, can be used.

In a preferred embodiment, the UV disinfectant light providing light source is embedded in a retrofit downlight which provides light with a wavelength of both 222 nm Far UV-C for disinfection purposes and general illumination. Preferably, the system further comprises a motion sensor in cases in which a light source is used that emits light which can pose a health risk to living beings. In this embodiment, the motion sensor is adapted to detect the presence of a living being in the space lighted by the light source and the controller is adapted to control the light source such that disinfectant light is only provided when no motion, particularly no presence, of a living being, is detected in the space. Moreover, in order to preserve the longevity of a Far UV-C light source, during periods of inactivity in the lighted space, for instance, when no people have visited the space in the last 5 hours and hence less disinfection is required, the controller can be adapted to control the light source to apply the disinfectant light with a lower radiation dose, for instance, for a shorter time period. Such downlights can be applied to target disinfection applications in hospitals, clinics, assisted living communities, as well as schools and childcare centers. In another application, the system can be embedded in a corridor or free-standing portal placed at the entrance of a shared space to disinfect the surface of an object upon passing through the space.

Moreover, although not as effective for containing viruses, also light in the infrared spectrum can be employed for disinfection purposes with respect to certain pathogens. Thus, based on the application, the light source can also be adapted to use light in the infrared spectrum as disinfectant light.

Although in above embodiments the controller is shown integrated with the light source, i.e. in the same housing of the light source, in other embodiments the controller can be provided external to the housing of the light source and in wired or wireless communication with the light source for controlling the same. Moreover, the controller can also be part of an overall controller adapted to control a plurality of light sources and/or other devices.

Although in the above embodiments, the disinfection status providing unit is shown integrated with the detection surface, i.e. provided in the same housing, the disinfection status providing unit can also be provided external to a housing of the detection surface and in wired or wireless communication with the detection surface to receive the result of the detection of the detection surface. Moreover, the disinfection status providing unit can also be provided as part of an overall disinfection status providing unit adapted to determine the disinfections status for a plurality of sensor devices or even as part of an overall controller as described above. Furthermore, the disinfection status deriving unit can be provided integrated into the lighting unit, for instance, integrated into a controller of the lighting unit.

Although in the above embodiments, the light unit was provided on a ceiling of a room and as part of the environmental lighting of the room, in other embodiments the light unit can be a dedicated light unit that only provides disinfectant light. Moreover, the light unit can be provided as part of a disinfectant device, for instance, a handheld disinfectant device that can be used similar to a flashlight for disinfecting small objects or small parts of a surface.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the controlling of the light source, the deriving of the disinfection status, etc., performed by one or several units or devices can be performed by any other number of units or devices. These procedures can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

The invention relates to a system, a light unit, a sensor device and a method for disinfection applications. The system comprises a) a light unit comprising i) a light source, e.g. LED, adapted to emit at least disinfectant light, and ii) a controller for controlling the light source such that at least a part of the light is encoded with information on light characteristics of the disinfectant light, and b) a sensor device comprising i) a detection surface adapted to be arranged on an object and to detect the encoded light, and ii) a disinfection status deriving unit adapted to derive a disinfection status of the object based on the light characteristics encoded in the encoded light. This allows for providing a good estimate of a disinfection status of an object treated with the disinfection light.

1. System for disinfection applications, wherein the system comprises:
a light unit comprising:
a light source adapted to emit light, wherein at least a part of the emitted light is disinfectant light usable for disinfection purposes, wherein the light source, is adapted to emit light with a wavelength in the ultraviolet spectrum, infrared part of the spectrum or in the purple part of the visible spectrum as disinfectant light, and
a controller configured to control the light source, wherein the controller is configured to control the light source such that at least a part of the emitted light is encoded with a digital code indicative of information on light characteristics of the emitted disinfectant light, wherein the information on light characteristics comprises information indicative of a light spectrum, intensity and/or beam characteristics of at least the part of the emitted disinfectant light that is usable for disinfection purposes, and
a sensor device comprising:
a detection surface adapted to be arranged on an object and to detect emitted light from the light unit, wherein the detection surface is adapted to detect at least the emitted encoded light, and
a disinfection status deriving unit adapted to derive a disinfection status of the object based on the light characteristics encoded in the emitted encoded light, said disinfection status being indicative of an expected contamination of the object with predetermined gems.

2. The system according to claim 1, wherein the disinfection status deriving unit is further adapted to determine a time of exposure of the object indicative of the time the object has been illuminated by the disinfectant light based on the light detected by the detection surface and to derive the disinfection status of the object further based on the time of exposure.

3. The system according to claim 1, wherein the sensor device further comprises an object characteristics providing unit adapted to provide information on characteristics of the object to which the sensor device is attached, wherein said characteristics comprising at least one of: a type of the object, a location of the object, a general structure of the object, an orientation of the object, and wherein the disinfection status deriving unit is adapted to further derive the disinfection status of the object based on the object characteristics.

4. The system according to claim 1, wherein the system further comprises a feedback providing unit adapted to provide feedback information from the sensor device to the light unit, wherein the feedback information is indicative of the disinfectant light received by the object, the disinfection status of the object and/or the object characteristics, wherein the controller is further adapted to control the light source based on the feedback information.

5. The system according to claim 3, wherein the object characteristics providing unit is adapted to provide information on characteristics of the object, the characteristics comprising usage information indicative of a usage of the object in a time period since a last disinfection, wherein the usage information is provided such that from the usage information an expectable contamination can be estimated and wherein, the disinfection status unit is adapted to further derive the disinfection status based on the usage information.

6. The system according to claim 3, wherein the sensor device further comprises a biosensor unit adapted to detect biological components contaminating the object, and wherein the object characteristics comprise information indicative of a detection result of the biosensor.

7. The system according to claim 4, wherein the controller is further adapted to determine based on the feedback information a measure for the effectiveness of the disinfection and to control the light source based on the determined effectiveness measure, wherein said effectiveness measure is indicative of an increase of the disinfection status.

8. (canceled)

9. The system according to claim 7, wherein the controller is adapted to control the light source such that the light is encoded using a low frequency modulation or by switching the light on and off.

10. The system according to claim 1, wherein the sensor device further comprises an output unit adapted to provide an output signal indicative of the derived disinfection status of the object.

11. The system according to claim 1, wherein the controller is configured to control the light source such that the disinfectant light is encoded with the information on light characteristics of the emitted disinfectant light, and/or wherein the light source further emits additional light with a wavelength below the ultraviolet spectrum in addition to the disinfectant light, wherein the light source is controlled by the controller such that the additional light is encoded with the information on light characteristics of the emitted disinfectant light.

12. A light unit for use in a system according to claim 1, the light unit comprising:
a light source adapted to emit light, wherein at least a part of the emitted light is disinfectant light usable for disinfection purposes, and
a controller configured to control the light source, wherein the controller is configured to control the light source such that at least a part of the emitted light is encoded with a digital code indicative of information on light characteristics of the emitted disinfectant light.

13. A sensor device for use in a system according to claim 12, the sensor device comprising:
a detection surface adapted to be attached to an object and to detect emitted light from a light unit as disclosed in claim 12, wherein the detection surface is adapted to detect at least the emitted encoded light, and
a disinfection status deriving unit adapted to derive a disinfection status of the object based on the light characteristics encoded in the emitted encoded light, said disinfection status being indicative of an expected contamination of the object with predetermined gems.

14. A method for disinfection applications, wherein the method comprises the steps:
providing a light source adapted to emit light, wherein at least a part of the emitted light is disinfectant light usable for disinfection purposes, wherein the light source is adapted to emit light with a wavelength in the ultraviolet spectrum, infrared part of the spectrum or in the purple part of the visible spectrum as disinfectant light, and
controlling the light source such that at least a part of the emitted light is encoded with a digital code indicative of information on light characteristics of the emitted disinfectant light, wherein the information on light characteristics comprises information indicative of a light spectrum, intensity and/or beam characteristics of at least the part of the emitted disinfectant light that is usable for disinfection purposes, and
arranging a detection surface on an object,
detecting using the detection surface the emitted encoded light from the light source, and
deriving a disinfection status of the object based on the light characteristics encoded in the emitted encoded light, said disinfection status being indicative of an expected contamination of the object with predetermined gems.
